(19) United States
(12) Patent Application Publication
(10) Pub. No.: US 2002/0198258 A1
(43) Pub. Date: Dec. 26, 2002

(54) Therapeutic substances and methods of making and using same

(76) Inventors: Beverly Ann Brown, Dallas, TX (US); Gary Osborn, Dallas, TX (US)

Correspondence Address: John D. Kaufmann, Jackson Walker, LLP, Suite 600, 2435 N. Central Expressway, Richardson, TX, 75080, US

(21) Appl. No.: 09/934,779

(22) Filed: Aug. 22, 2001

Related U.S. Application Data
(60) Provisional application No. 60/291,489, filed on May 16, 2001.

(51) Int. Cl. A61K 9/127; A61K 31/24

(52) U.S. Cl. 514/535; 424/450

(57) ABSTRACT

A basic therapeutic unit comprises procaine hydrochloride encapsulated by a liposome. A quantity of the units are suspended in a cream or emollient carrier so that the therapeutic units may be topically applied to the skin for penetration therethrough and through subjacent blood vessel walls and entry into the circulatory system, following which the procaine and its metabolites pass the blood/brain barrier.

This application is related to and based on Provisional U.S. Application Serial No. 60/291,489, filed May 16, 2001.

### FIELD OF THE INVENTION

The present invention relates to therapeutic substances and methods of making and using same, and, more particularly, to a therapeutic unit which serves as the basis for topical procaine-delivery systems that eliminate the need for injecting procaine, and to methods for making and using these procaine delivery systems.

### BACKGROUND OF THE INVENTION

Procaine hydrochloride (“procaine”) and its ability to act as a local anesthetic were discovered by Dr. Alfred Einhorn circa 1905. On Feb. 13, 1906, Dr. Einhorn received U.S. Pat. No. 812,554 covering procaine. Procaine is C₁₃H₂₀N₂O₂ HCl [NH₂C₆H₄COOCH₂CH₂N(C₂H₅)₂.HCl] or 2-(diethylamino)ethyl ester 4-aminobenzoic acid HCl.

In the U.S., where procaine is called “Novocain,” it is still used by dentists for anesthetic purposes. Following some early reports that patients receiving procaine had experienced beneficial side effects apart from procaine's anesthetic activity, research into therapeutic uses of procaine began in the 1920's. Papers reported that direct hypodermic injection of procaine into joints and muscles benefited people suffering from a range of ailments.

In 1949, Dr. Ana Aslan conducted tests of the beneficial activity of injected procaine. While she claimed that positive results were achieved, she discovered that an enzyme, pseudo-cholinesterase, degraded procaine in a short time within the human body, thus necessitating frequent injections to produce benefits. In 1951 Dr. Aslan added various agents to procaine to eliminate its anesthetic effects and to stabilize it within the human body for up to 6 hours, calling the mixture “Gerovital H3” or “GH3.” In descending order of prevalence, GH3 includes procaine hydrochloride, ascorbic acid, citric acid, benzoic acid, potassium metabisulphite and disodium phosphate.

Various reports and observers have attributed to procaine delivered to the circulatory system by hypodermic injection the following beneficial effects: significantly fewer episodes of illness; depression alleviation; alleviation of paroxysmal supraventricular tachycardia, anterial fibrillation and complete heart block; improvement in skin condition, sleep patterns, blood pressure and heart arrhythmia; cessation or reversal of hair loss/graying and wrinkling/hardening of the skin; stimulation of the body to produce vitamin K, folic acid, choline, acetylcholine and thiamin; and anti-aging. Procaine has been used to treat arthritis; migraine headache; abnormal (high or low) blood pressure; peptic ulcers; acne; Parkinson's disease; Hodgkin's disease; low sex drive; multiple sclerosis; arteriosclerosis; sickle cell anemia; hypoglycemia; hyper- and hypo-tension; diabetes; herpes; senility; rheumatism; poor hearing; poor eyesight; failing memory; muscle fatigue; bad circulation; angina pectoris; osteoporosis; impotence; frigidity; liver spots; varicose veins; psoriasis; wrinkles; asthenia; edema; emphysema; neuralgia; and excessive cholesterol.

As discussed above, it is known to deliver procaine to the circulatory system of the human body by hypodermic injection. It is also known to deliver procaine to the body by ingestion, see U.S. Pat. Nos. 5,162,344; 5,254,686; and 5,283,258. Taking procaine by injection effects more immediate results than does ingestion, with the former resulting in the procaine passing the blood-brain barrier almost immediately. Ingesting procaine in the form of a tablet appears to produce a delayed beneficial results because of the slower rate of absorption of ingested procaine. Ingestion does, however, produce another beneficial effect, namely stimulation of the intestinal tract to function more efficiently and to produce more vitamins and folic acid.

Whether taken by injection or orally, procaine in the body is metabolized and hydrolyzed, either in the blood or in the intestine, into its metabolytes, two water soluble moieties, para-aminobenzoic acid (“PABA”; C₇H₇O₂N, part of the folic acid molecule) and diethylaminoethanol (“DEAE”), a neurotransmitter. Administration to humans of these two metabolytes separately does not appear to produce the same beneficial effects as does procaine. It is theorized that procaine as such, not merely its two uncombined, separate metabolytes, must pass into the circulatory system and thereafter metabolyze or hydrolyze into PABA and DEAE within the blood for there to occur beneficial effects.

Administration of procaine by injection is not ideal. Typically, several milliliters—5 or so—must be injected into the hip several times a week. Injections must be rather deep into the tissue—about 1½ inch—for beneficial effects to be experienced. There are reports that, notwithstanding the realization of beneficial effects, after 9 months of such painful injections, it was nearly impossible to tolerate further injection due to scar buildup in the hip area.

A primary goal of the present invention is to provide a pain-free, less drastically invasive alternative to administering procaine by injection which achieves the rapid, beneficial results of procaine by injection.

### SUMMARY OF THE INVENTION

In order to achieve the foregoing goal, the present invention in its broadest aspect contemplates a “basic therapeutic unit,” specifically, an aqueous solution of procaine hydrochloride associated with a carrier which functions as a tissue-penetration enabler. The carrier transports the procaine through the skin and subjacent blood vessels so that the procaine may enter the blood and thereafter pass the blood/brain barrier. Within the blood, some of the procaine apparently metabolizes or hydrolyzes into its two metabolytes, PABA and DEAE. It is theorized that some of the procaine, as well as previously metabolized or hydrolyzed procaine, also passes the blood/brain barrier. In any event, the procaine and its metabolytes enter the blood and pass the blood/brain barrier, thereby effecting the above-described health benefits.

In one specific preferred embodiment, the carrier is a liposome and the procaine hydrochloride is encapsulated therewithin. A liposome includes a microscopic, generally spherical pouch, sac or bubble (diameter typically about 100 nm). The spherical pouch is defined by a membrane comprised of layers of lipid (phospholipid) molecules (the “bilayer”) surrounding an aqueous core. Water soluble substances may be encapsulated within the bilayer. Substances that are not easily dissolved in water may be incorporated into the bilayer itself. The liposome is able to pass through and between the cells of the skin and the subjacent blood vessels and into the blood stream, so that the procaine and/or its metabolytes, PABA and DEAE, is carried throughout the body. The procaine enters the blood and therewithin at least some of it metabolizes or hydrolyzes into PABA and DEAE. Further, PABA, DEAE and procaine pass the blood/brain barrier, whereat further procaine is metabolized or hydrolyzed into PABA and DEAE. It is theorized that the metabolization or hydrolysis of procaine into PABA and DEAE within the blood and the brain stimulate the production therewithin of other beneficial substances.

In another aspect of the present invention, there is provided a therapeutic formulation which is comprised of a plurality of the procaine-containing basic therapeutic units held in suspension in a medium—for example an emollient or cream—which is suitable for topical application to the skin. Contained within the liposome membrane along with the encapsulated procaine—or incorporated into the bilayer itself—may be other therapeutic substances intended to effect benefits in addition to, or to enhance or supplement the action of, the procaine. Similarly, therapeutic substances may also be added to, and held in suspension by, the emollient or cream.

In other aspects, the present invention contemplates methods of making the basic procaine-containing therapeutic units, the topical therapeutic formulation containing the units, and the topical mixture containing liposome-encapsulated procaine as well as other therapeutic substances contained within the membrane or incorporated into the bilayer.

Lastly, the present invention contemplates methods of using the basic procaine-containing units, the tropical mixture containing the units and the topical mixture including the basic units that contain procaine as well as other therapeutic substances encapsulated by the bilayer or incorporated into the bilayer.

### DETAILED DESCRIPTION

The discovery of the anesthetic procaine and it later discovered therapeutic effects have been discussed above. The present invention in its broadest aspect comprises an aqueous solution of procaine associated with a carrier that is skin-penetrating and blood vessel-penetrating. This association of procaine and the carrier is referred to herein as a “basic therapeutic unit.” The basic therapeutic unit is suitable for topical application to the skin, following which the carrier and the procaine pass through the skin and the subjacent blood vessels so that the procaine enters the blood cells and subsequently passes the blood/brain barrier, as described earlier.

In preferred embodiments, the carrier is a liposome, with a quantity of procaine being encapsulated within the membrane or bilayer of each liposome sphere. The liposome/procaine association is referred to herein as the “basic liposome therapeutic unit.” As is known, depending on the properties of the liposomes, the liposomes may transport the procaine to the blood cells, as described earlier, as well as to selected body sites.

Liposomes were first produced in 1961 by Alec D. Bangham, who found that phospholipids combined with water immediately formed spherical bodies having the water-encapsulated-in-membrane structure described above. Specific liposomes after passing through the skin may pass also through the walls of blood vessels or may attach to cellular membranes Some liposomes appear to fuse with cells, releasing their contents into the cells. Other liposomes are taken up by cells and their membranes are incorporated into the cell membranes, while the encapsulated contents of the liposome are released within the cell. According to the present invention, the spherical membrane or bilayer of the liposomes encapsulate procaine.

Where, as here, the goal is to have procaine generally available in the circulatory system, the preferred liposomes are those made from soy lecithin. When the soy lecithin is added to water containing dissolved crystals of procaine HCl, the liposomes form and encapsulate an aqueous solution of procaine. The procaine-containing liposomes can be topically applied to the skin and the liposomes and their encapsulated procaine will pass through the skin and the subjacent blood vessels, thereafter entering the blood cells.

These procaine-containing liposomes may be produced by dispersing granular soy lecithin and an emulsifier, such as polysorbate 80, in heated (60°-70° C.) sterile water in which procaine hydrochloride has been dissolved, followed by continuous mixing until homogeneous. The proportions of these materials in approximate weight percent are:

| | |
|---|---|
| procaine | 25.5% |
| water | 70.7% |
| soy lecithin | 1.3% |
| polysorbate 80 | 2.5% |

A mass of liposomes feels oily or greasy and is not pleasing to the touch or to areas of the skin to which it is applied. In preferred embodiments, the esthetics and “feel” of the topically applied liposomes may be improved by an emollient or cream in which the liposomes are suspended to thereby produce a topical therapeutic formulation. Specifically, the “feel” and cosmetic acceptability of the liposomes may be enhanced by suspending the liposomes in anhydrous vanishing cream to which there may be also added a preservative and a thickener. The vanishing cream and the preservative, preferably butylated hydroxytoluene, are heated until a clear melt is obtained. The thickener, preferably xanthan gum, is then added and mixing is effected until a homogeneous mixture is obtained.

Thereafter, the first liposome-containing mixture and the mixture containing the vanishing cream are admixed while warm and are stirred continuously until room temperature is reached. The resulting therapeutic formulation with the procaine-containing liposomes suspended in a vanishing cream is esthetically pleasing and not unpleasant to apply topically to the skin. Moreover, it has been found that the addition of a dispersant, such as simethicone, and a minor amount of peppermint essential oil, render the therapeutic formulation pleasing to the touch and smell.

The proportions of the materials in the therapeutic formulation by approximate weight percent are:

| | | | |
|---|---|---|---|
| procaine | 20% | vanishing cream | 18.3% |
| water | 55.8% | butylated hydroxytoluene | .1% |
| lecithin | 1% | xanthan gum | 1.5% |
| polysorbate 80 | 2% | simethicone | .5% |
| | | peppermint oil | .8% |

Clearly, formulations other than those set forth above may be used. The important event is the association of procaine with a carrier, such as a number of liposomes, by which the an effective amount of intact procaine, as well as its metabolites, can enter the body and the cells of targeted body sites, such as blood cells and brain cells and other cells. As noted earlier those having ordinary skill in the liposome art are able to design and formulate liposomes which can target specific organs or parts of the body. That being the case, procaine may, accordingly, be delivered to specified body sites for hydrolysis within the cells found at those sites. Adjusting the amount and type of vanishing cream, thickener, preservative, emulsifier and dispersant, as well as a decision to use or not use peppermint oil or some other similar substance, is within the skill of the art. Any formulation that is pleasing to the touch and smell and which, when topically applied, allows the procaine-carriers or liposomes to deliver their encapsulated procaine for passage through the skin and other body tissue to reach target cells is within the scope of the present invention and the appended claims. Stated differently, any procaine-containing carrier, such as a liposome, which is a basic therapeutic unit or a basic liposome therapeutic unit, both defined above, falls within the scope of the present invention. Whatever else may be added to these basic units, the mere presence thereof, without more, is sufficient to fall under the umbra of the present invention, as expressed in the claims hereof.

Another therapeutic formulation, similar to the previous formulation, contains all of the ingredients thereof, and in addition contains vitamins, such as aminobenzoic acid (a B vitamin) and cyanocobalamin (vitamin B-12); a neurotransmitter, such as NADH (nicotinamide adenine dinucleotide); an antifungal, such as potassium sorbate; a homeopathic human growth hormone; and a compatible pH adjuster such as sodium phosphate dibasic heptahydrate. It is presently unknown whether the foregoing substances are incorporated into the interior of the liposome, into the dilayer of the liposome or are otherwise introduced into the circulatory system

Tests of the above therapeutic formulations have confirmed that topical application thereof to the skin results in both the entry of procaine and its metabolytes into the blood and passage of the procaine and its metabolytes through the blood/brain barrier. Nevertheless, a technique has been developed for accentuating and amplifying the rate at which procaine is delivered to the blood.

Specifically, it has been found that the addition of a vasodilator, such as methyl nicotinate in a weight percent of about 0.3%, to the therapeutic formulations increases the rate at which procaine is delivered to the blood cells following topical application of the therapeutic formulation to the skin.

Those having skill in the art will appreciate that the main thrusts of this invention arise from the basic therapeutic unit and the basic liposome therapeutic unit, as well as from therapeutic formulations containing same, all as described above and as set forth in the following claims.

What is claimed is:

1. A basic therapeutic unit for topical application to the skin, comprising:
a quantity of an aqueous solution of procaine hydrochloride associated with a carrier which functions as a tissue-penetration enabler

2. The basic therapeutic unit of claim 1, wherein the carrier is a skin-penetration enabler and a blood vessel-penetration enabler and the basic therapeutic unit enters the blood after it penetrates and passes through the skin and subjacent blood vessels, whereafter the procaine and/or its metabolytes then pass the blood/brain barrier.

3. The basic therapeutic unit of claim 1, wherein the carrier transports the procaine through the skin and subjacent blood vessels and into the blood, and the procaine thereafter passes the blood/brain barrier.

4. The basic therapeutic unit of claim 3, wherein the carrier delays metabolization and hydrolysis of the procaine so that both procaine and its metabolytes are present in the blood and pass the blood/brain barrier.

5. The basic therapeutic unit of claim 4, wherein the carrier is a liposome encapsulating therewithin the quantity of procaine hydrochloride.

6. The therapeutic formulation including the basic therapeutic unit of claim 5, which comprises a plurality of the basic therapeutic units suspended in a medium which is suitable for topical application to the skin.

8. The basic therapeutic unit of claim 1, wherein the carrier is a liposome encapsulating therewithin the quantity of procaine hydrochloride.

9. The therapeutic formulation including the basic therapeutic unit of claim 8, which comprises a plurality of the basic therapeutic units suspended in a medium which is suitable for topical application to the skin.

10. The therapeutic formulation of claim 9, which further comprises a vasodilator in the medium.

11. A basic therapeutic unit for topical application to the skin, comprising:
a quantity of an aqueous solution of procaine hydrochloride encapsulated within a liposome.

12. The basic therapeutic unit of claim 11, which further comprises one or more substances, in addition to the procaine, that are encapsulated within the liposome.

13. The therapeutic formulation including the basic therapeutic unit of claim 11, which comprises a plurality of the basic therapeutic units and a medium that holds the plural medication units in suspension and is suitable for topical application to the skin.

14. The therapeutic formulation unit of claim 13, which further comprises one or more substances that, in addition to the basic therapeutic units, are held in suspension in the medium.

15. The therapeutic formulation of claim 14, which further comprises one or more substances, in addition to the procaine, that are encapsulated within the liposome.

16. The therapeutic formulation of claim 14, wherein a vasodilator is present as an additional substance.

17. The therapeutic formulation of claim 16, wherein the vasodilator is methyl nicotinate.

18. The therapeutic formulation of claim 14, wherein present as additional substances in the medium are one or more of a vasodilator, a vitamin, a neurotransmitter, a pH adjuster, a thickener, a preservative, an emulsifier, an anti-fungal, a dispersant, a growth hormone, and sterile water.

19. The therapeutic formulation of claim 18, wherein the vasodilator is methyl nicotinate.

20. The basic therapeutic unit of claim 11, which further comprises a vasodilator.

21. The therapeutic formulation including the basic therapeutic unit of claim 20, which comprises a plurality of the basic therapeutic units and a medium that holds the therapeutic units and the vasodilator in suspension and is suitable for topical application to the skin.

22. The therapeutic formulation of claim 21, wherein the liposomes and the vasodilator facilitate and enhance the penetration and passage of the basic therapeutic units through the skin and subjacent blood vessels to permit the procaine hydrochloride to pass through the skin and the blood vessels and the procaine and its metabolytes to then circulate with the blood and thereafter pass the blood/brain barrier.

23. The therapeutic formulation of claim 22, wherein each liposome suppresses and delays metabolization and hydrolysis of the procaine after it and the procaine are present in the blood.

24. The therapeutic formulation of claim 21, wherein the medium is an emollient or cream.

25. The therapeutic formulation of claim 24, wherein the medium is vanishing cream.

26. The therapeutic formulation of claim 11, wherein the membrane of the liposome is a plurality of molecules of soy lecithin.

27. A method of making a basic therapeutic unit, which comprises:
encapsulating an aqueous solution of procaine hydrochloride within a liposome.

28. The method of making a therapeutic formulation from the basic therapeutic unit of claim 27, which comprises suspending a plurality of the basic therapeutic units in a medium that is suitable for topical application to the skin.

29. The method of claim 28, wherein the medium is an emollient or cream.

30. The method of claim 28, which further comprises encapsulating within the liposome substances in addition to procaine.

31. The method of claim 28, which further comprises suspending in the medium substances in addition to the basic therapeutic units.

32. The method of claim 31, which further comprises encapsulating within the liposome substances in addition to procaine.

33. A method of beneficially affecting the human body, which comprises:
encapsulating an aqueous solution of procaine hydrochloride within a liposome; and
applying the liposome to the skin, so that the liposome passes through the skin and the blood vessels and the procaine thereafter enters the blood stream to produce beneficial effects.

34. The method of claim 33, wherein the liposome-applying step is effected by applying to an area of the skin a medium which is suitable for topical application to the skin and which holds in suspension therein a plurality of procaine-encapsulating liposomes.

35. The method of claim 34, wherein the medium is an emollient or cream.

36. The method of claim 35, wherein the medium is vanishing cream.

37. The method of claim 34, wherein a vasodilator is also applied to the area of the skin.

38. The method of claim 37, wherein the vasodilator is held in suspension within the medium.

39. The method of claim 38, wherein the vasodilator is methyl nicotinate.

40. The method of claim 39, wherein the medium is vanishing cream.

41. A method of making a basic therapeutic unit, which comprises:
dispersing polysorbate 80 liquid and granulated soya lecithin in heated sterile water and mixing until the mixture is homogeneous;
heating vanishing cream and butylated hydroxytoluene until a clear melt is produced; and
adding together the homogeneous mixture and the clear melt and mixing continuously until at room temperature.
